(19) United States
(12) Patent Application Publication
(10) Pub. No.: US 2023/0108071 A1
(43) Pub. Date: Apr. 6, 2023

(54) SYSTEMS AND METHODS FOR SELF-TRACKING REAL-TIME HIGH RESOLUTION WIDE-FIELD OPTICAL COHERENCE TOMOGRAPHY ANGIOGRAPHY

(71) Applicant: OREGON HEALTH & SCIENCE UNIVERSITY, Portland, OR (US)

(72) Inventors: Yali Jia, Portland, OR (US); Xiang WEI, Portland, OR (US)

(73) Assignee: OREGON HEALTH & SCIENCE UNIVERSITY, Portland, OR (US)

(21) Appl. No.: 17/796,066

(22) PCT Filed: Jan. 29, 2021
(86) PCT No.: PCT/US2021/015882

§ 371 Date: Jul. 28, 2022

Related U.S. Application Data
(60) Provisional application No. 62/968,894, filed on Jan. 31, 2020.

(51) Int. Cl. A61B 3/10; A61B 5/00

(52) U.S. Cl. CPC A61B 3/102; A61B 5/0066; G06T 2207/10101

(57) ABSTRACT

Disclosed herein are methods and systems for self-tracking of motion artifacts (e.g., due to eye blinking and/or micro saccades motion) in optical coherence tomography (OCT) and/or OCT angiography (OCTA). A motion strength index (MSI) may be determined based on OCTA data, and the presence of excessive motion may be determined based on the MSI (e.g., if the MSI is greater than a threshold). The system may re-acquire OCT data at scan locations for which excessive motion is detected. A cross (X) scanning pattern for alignment is also described. Other embodiments may be described and claimed.

### CROSS REFERENCE TO RELATED APPLICATION

This application claims priority to U.S. Provisional Pat. Application No. 62/968,894, titled “SYSTEMS AND METHODS FOR SELF-TRACKING REAL-TIME HIGH RESOLUTION WIDE-FIELD OPTICAL COHERENCE TOMOGRAPHY ANGIOGRAPHY,” which was filed Jan. 31, 2020.

### FIELD

Generally, the field involves methods of imaging using optical coherence tomography. In particular, the field involves methods of self-tracking real-time high resolution wide-field optical coherence tomography angiography (OCTA).

### GOVERNMENT INTEREST STATEMENT

This invention was made with government support under R01 EY027833 awarded by the National Institutes of Health. The government has certain rights in the invention.

### BACKGROUND

Optical coherence tomography (OCT) is a non-invasive imaging modality capable of exceptional measurements of retinal and choroidal structure. In contrast to fundus photography, OCT can provide high-resolution, three-dimensional structural information. OCT is also able to procure angiographic data (OCT angiography (OCTA)) by measuring the inherent motion contrast between successive OCT images. Compared to conventional angiographic imaging techniques such as fluorescein angiography (FA), OCTA provides superior resolution as well as volumetric data. As a non-invasive imaging method, OCTA also avoids the potential side effects and discomfort associated with dye injection techniques.

Wide-field OCTA imaging has recently attracted much research interest. Some diseases, like diabetic retinopathy (DR), often have early stage peripheral biomarkers not visible in macular scans. Detection and treatment in the early stage can potentially slow or stop the disease from further progression. Because of potential side effects, FA is not suitable for routine imaging. In contrast, wide-field OCTA can be widely used, and therefore stands a better chance of catching pathological developments early. Wide-field OCT has been studied since 2010, but only large vessels can be resolved in the OCT images. With the increase of laser speed and image processing techniques, high-resolution wide-field OCTA has been explored by several research groups. Wide-field OCT systems have a much larger field-of-view (FOV) compared to conventional systems, so in order to maintain image resolution, the total number of sampling points needs to be increased along both fast and slow axes. Typically, the sampling density should meet the Nyquist criterion. This leads to longer inter-frame and total imaging times, which in turn means that OCTA artifacts due to microsaccidic motion, blinking, and tear film evaporation will be more prominent.

Bleeding edge, large FOV systems therefore require some means of artifact compensation. Broadly, artifacts can be removed during postprocessing and/or they can be eliminated during OCTA acquisition. Using the former approach (postprocessing), image processing algorithms can be used to suppress some artifacts. However, postprocessing corrections involve some degree of information loss and may need multiple volume acquisitions, resulting in doubled or tripled total imaging time. Another common approach for constructing large FOV OCTA images is to collect several smaller, high-resolution images and construct the full image by montage. However, image stitching can introduce new artifacts. Instead of relying on postprocessing, instrumental improvements can yield high resolution, large FOV OCTA simply by increasing the scan rate, and such fast-scanning systems have been developed. Still, even in state-of-the-art systems data acquisition requires some trade-off between scan resolution and FOV size.

A final approach is to use real-time blink and motion artifact correction to suppress artifacts. Many prototypes and commercial OCT systems, such as RT-Vue (Optovue Inc., USA), CIRRUS (Carl Zeiss AG, Germany) and Spectralis (Heidelberg Engineering, Germany), incorporate motion tracking. Currently, such eye motion tracking systems are based on allied imaging methods, like infrared fundus photography or scanning laser ophthalmoscopy (SLO). To function, the additional imaging hardware must be coupled to the OCT optical axis and simultaneously acquiring images. This scheme has several drawbacks. Both the fundus camera and SLO images need to be acquired and processed separately from the OCT image, increasing the processing requirements of the system. In addition, SLO and fundus photography can only provide indirect information about the OCT image quality. Finally, including other imaging modalities in the OCT system increases system complexity, with all the attendant considerations (such as increased cost and more arduous and potentially frequent component repair).

### BRIEF DESCRIPTION OF THE SEVERAL VIEWS OF THE DRAWINGS

FIG. 1 is a flow chart illustrating a method of real-time OCT/OCTA image processing in accordance with various embodiments. One or more aspects of the method may be performed by a graphics processing unit (GPU). For example, the raw OCT spectrum is first transferred from the memory of a central processing unit (CPU) to a memory of the GPU. A GPU-based dispersion compensation algorithm is applied to the raw spectrum. Both the full OCT spectrum and split-spectrums for split-spectrum amplitude decorrelation algorithm (SSADA) are processed using a Fast Fourier Transform (FFT). An OCT image is generated after the FFT (e.g., immediately after, without intervening processing operations). An OCTA image is generated after application of a decorrelation algorithm. All the images may be transferred back to the CPU memory.

FIGS. 2A and 2B are OCTA mean projection en face images acquired from a healthy volunteer without the tracking system engaged. FIGS. 2C and 2D depict respective mean values from each OCTA B-scan. FIGS. 2E and 2F depict motion strength index (MSI) values calculated using the respective en face OCTA image, with line 202 indicating a threshold.

FIGS. 2G and 2H depict a motion trigger signal generated after the threshold is applied to the MSI value.

FIG. 3 is a flow chart of a self-tracking process, in accordance with various embodiments.

FIG. 4A is a diagram illustrating a cross-scanning pattern in accordance with various embodiments. Line 402 indicates the horizontal scan path, line 404 indicates the vertical scan path, and lines 406a-b indicates the scanner reset path. FIG. 4B is a plot of a voltage signal applied to the X and Y galvo scanner in accordance with the cross-scanning pattern in some embodiments. FIG. 4C is a horizontal cross-sectional image (e.g., x-scan) in accordance with various embodiments. FIG. 4D is a vertical cross-sectional image (e.g., y-scan) in accordance with various embodiments.

FIGS. 5A-5G illustrate high-density wide-field OCTA images in accordance with various embodiments. FIG. 5A is a wide-field OCTA image acquired without tracking engaged. FIG. 5B is a wide-field OCTA image acquired with self-tracking engaged. The vignetting artifact is caused by eye lashes. FIG. 5C is a high-resolution wide-field OCTA image acquired with self-tracking engaged. FIGS. 5D and 5F are 3×3 mm inner retinal images cropped from the image of FIG. 5C. FIGS. 5E and 5G are 3 × 3 mm inner retinal angiograms acquired using a commercial system.

FIG. 6 is an inner retinal OCTA image acquired from a patient with diabetic retinopathy (DR), in accordance with various embodiments.

FIG. 7 schematically shows an example system for real-time self-tracking OCT processing, in accordance with various embodiments.

FIG. 8 schematically shows an example of a computing system in accordance with the disclosure.

### DETAILED DESCRIPTION

Disclosed are methods and systems for self-tracking real-time high resolution wide-field optical coherence tomography (OCT) angiography (OCTA). Also disclosed herein is an exemplary system for acquiring and/or processing OCTA images. The exemplary system comprises an OCT device configured to acquire OCT structural and angiography data in functional connection with a computing device having a logic subsystem and data holding capabilities. In embodiments the computing device is configured to receive data from the OCT device and perform one or more operations of the methods described herein.

In some embodiments, a tracking method is described that does not require additional hardware. For example, the tracking method may be directly based on OCTA. Some OCTA algorithms calculate decorrelation, which can be used to detect motion. The tracking method described herein may overcome one or more of the problems with prior techniques, as described above.

In the following detailed description, reference is made to the accompanying drawings which form a part hereof, and in which are shown by way of illustration embodiments that can be practiced. It is to be understood that other embodiments can be utilized and structural or logical changes can be made without departing from the scope. Therefore, the following detailed description is not to be taken in a limiting sense, and the scope of embodiments is defined by the appended claims and their equivalents.

Various operations can be described as multiple discrete operations in turn, in a manner that can be helpful in understanding embodiments; however, the order of description should not be construed to imply that these operations are order dependent.

The description may use the terms “embodiment” or “embodiments,” which may each refer to one or more of the same or different embodiments. Furthermore, the terms “comprising,” “including,” “having,” and the like, as used with respect to embodiments, are synonymous.

In various embodiments, structure and/or flow information of a sample can be obtained using OCT (structure) and OCT angiography (flow) imaging-based on the detection of spectral interference. Such imaging can be two-dimensional (2-D) or three-dimensional (3-D), depending on the application. Structural imaging can be of an extended depth range relative to prior art methods, and flow imaging can be performed in real time. One or both of structural imaging and flow imaging as disclosed herein can be enlisted for producing 2-D or 3-D images.

Unless otherwise noted or explained, all technical and scientific terms used herein are used according to conventional usage and have the same meaning as commonly understood by one of ordinary skill in the art which the disclosure belongs. Although methods, systems, and apparatuses/materials similar or equivalent to those described herein can be used in the practice or testing of the present disclosure, suitable methods, systems, and apparatuses/materials are described below.

All publications, patent applications, patents, and other references mentioned herein are incorporated by reference in their entirety. In case of conflict, the present specification, including explanation of terms, will control. In addition, the methods, systems, apparatuses, materials, and examples are illustrative only and not intended to be limiting.

In order to facilitate review of the various embodiments of the disclosure, the following explanation of specific terms is provided:
A-scan: A reflectivity profile that contains information about spatial dimensions and location of structures within an item of interest. An A-scan is an axial scan directed along the optical axis of the OCT device and penetrates the sample being imaged. The A-scan encodes reflectivity information (for example, signal intensity) as a function of depth (z-direction).
B-scan: A cross-sectional tomograph that can be achieved by laterally combining a series of axial depth scans (i.e., A-scans) in the x-direction or y-direction. A B-scan encodes planar cross-sectional information from the sample and is typically presented as an image. Thus, a B-scan can be called a cross sectional image. The axis orthogonal to the A-scan axis in the plane of the cross-sectional scanning location of the B-scan is referred to as the fast axis. Accordingly, the scanner travels along the fast axis while obtaining A-scans that are combined to form one B-scan. The axis orthogonal to the plane of the cross-sectional scanning location of the B-scan is referred to as the slow axis.
Dataset: As used herein, a dataset is an ordered-array representation of stored data values that encodes relative spatial location in row-column-depth (x-y-z axes) format. In the context of OCT, as used herein, a dataset can be conceptualized as a three dimensional array of voxels, each voxel having an associated value (for example, an intensity value, a complex value having both amplitude and phase information, a decorrelation value, or other signal representations). An A-scan corresponds to a set of collinear voxels along the depth (z-axis) direction of the dataset; a B-scan is made up of set of adjacent A-scans combined in the row or column (x- or y- axis) directions. Such a B-scan can also be referred to as an image, and its constituent voxels referred to as pixels. A collection of adjacent B-scans can be combined form a 3D volumetric set of voxel data referred to as a 3D image. In the system and methods described herein, the dataset obtained by an OCT scanning device is termed a “structural OCT” dataset whose values can, for example, be complex numbers carrying intensity and phase information. This structural OCT dataset can be used to calculate a corresponding dataset termed an “OCT angiography” dataset reflecting flow within the imaged sample. There is a correspondence between the voxels of the structural OCT dataset and the OCT angiography dataset. Thus, values from the datasets can be “overlaid” to present composite images of structure and flow (e.g., tissue microstructure and blood flow) or otherwise combined or compared.
En Face angiogram: OCT angiography data can be presented as a 2D projection of the three dimensional dataset onto a single planar image called an en face angiogram. Construction of such an en face angiogram requires the specification of the upper and lower depth extents that enclose the region of interest within the retina OCT scan to be projected onto the angiogram image. These upper and lower depth extents can be specified as the boundaries between different layers of the retina (e.g., the voxels between the inner limiting membrane and outer plexiform layer could be used to generate an en face angiogram of the inner retina). Once generated, the en face angiogram image may be used to quantify various features of the retinal vasculature as described herein. This quantification typically involves the setting of a threshold value to differentiate, for example, the pixels that represent flow within vasculature from static tissue within the angiogram. These en face angiograms can be interpreted in a manner similar to traditional angiography techniques such as fluorescein angiography (FA) or indocyanine green (ICG) angiography, and are thus well-suited for clinical use. It is also common to generate en face images from structural OCT data in a manner analogous to that used to generate en face angiograms. Angiograms from different layers may also be color-coded and overlaid to present composite angiograms with encoded depth information; structural en ƒace images may also be included in such composite image generation.

Functional OCT, as used herein, broadly refers to the extension of OCT techniques to provide information beyond structural characterization. For example, whereas structural OCT imaging may be used to gather spatial information about a tissue’s anatomical organization, functional OCT may be used to gather information about processes occurring within that tissue sample such as blood flow, tissue perfusion and oxygenation, birefringence, etc. Examples of functional OCT include, but are not limited to, OCT angiography (OCTA) and associated techniques for characterizing blood flow, Doppler OCT, polarization-sensitive OCT, OCT elastography, spectroscopic OCT, differential absorption OCT, and molecular imaging OCT.

Embodiments herein provide a self-tracking method that suppresses eye motion and blinking artifacts on wide-field OCTA. In some embodiments, the method may be implemented without requiring hardware modification. A highly efficient graphic processing unit (GPU)-based, real-time OCTA image acquisition and processing technique may be used to detect eye motion artifacts. The algorithm may include an instantaneous motion index that evaluates the strength of motion artifact on en face OCTA images. Areas with suprathreshold motion and eye blinking artifacts may be automatically rescanned in real-time. Both healthy eyes and eyes with diabetic retinopathy were imaged using this system to test the techniques described herein. The disclosed tracking system can remove the blinking artifacts and large motion effectively.

Various aspects of the disclosed techniques are described in more detail below. For example, a system configuration for self-tracking real-time high resolution wide-field OCTA is shown and described. Additionally, in an aspect of the disclosed techniques, OCTA data is used to detect motion artifacts (e.g., eye blink and/or micro saccades motion). The motion signal generated by OCTA is reliable and accurate, and no additional imaging modality is needed. In some embodiments, the self-tracking method may be performed by a GPU. The GPU may enable real-time processing and tracking of motion artifacts.

In an aspect, an instantaneous motion strength index (MSI) is defined to evaluate motion. The MSI value may be compared to a threshold to determine whether the severity of the motion warrants re-scanning of the affected area. The MSI may be normalized so the effect of signal strength and/or system sensitivity is removed or reduced.

In another aspect, a cross-scanning pattern for scanning the sample with the OCT light source is described herein. The cross-scanning pattern may help the operator of the OCT system to align the OCT system/light source with the sample (e.g., retina), for example to avoid vignetting and/or other unwanted artifacts.

### Example Swept-Source OCT (SS-OCT) System

The techniques described herein may be implemented on any suitable OCT system. In one example, a customized 400-kHz swept-source laser is used in this system. The laser engine used in the examples and results described in this application is a swept wavelength laser source with a 400-kHz sweep rate (Axsun Technologies, USA), which is 4-6 times faster than the laser used in commercially available devices. The laser has a center wavelength of 1060 nm with 100 nm sweep range operating at 100% duty cycle. The maximum theoretical axial resolution is 4 µm in tissue. In some embodiments, the system may provide up to a 75-degree maximum FOV. For example, the system may use the optics design of sample arm described in Wei, Xiang, et al. “75-degree non-mydriatic single-volume optical coherence tomographic angiography.” Biomedical Optics Express 10.12 (2019): 6286-6295, hereby incorporated by reference herein. The spot size on the retina was calculated to be 15 µm, which is equivalent to the maximum lateral resolution that this system can achieve.

### GPU Based Real-Time OCT/OCTA

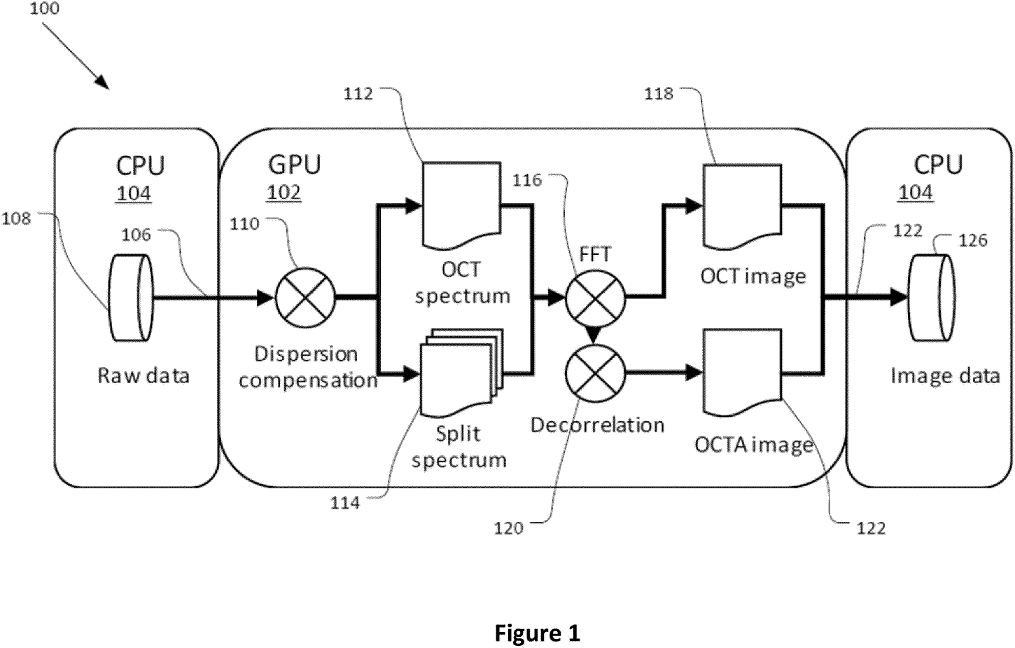

Various embodiments include a GPU-based real-time OCT/OCTA data acquisition and processing technique for a swept-source OCT system. Some or all aspects of the technique may be implemented by machine-readable instructions that are executed by the GPU. The split-spectrum amplitude-decorrelation angiography (SSADA) algorithm may be applied to compute OCTA flow signal. SSADA can increase the flow signal-to-noise ratio by combing flow information from each split-spectrum. The real-time processing efficiency can also be improved through GPU-based parallel data processing. To further improve the GPU processing efficiency, the OCT and OCTA images are processed in a single GPU thread (see FIG. 1, discussed further below). One of the major problems for real-time data processing is the data transfer speed, which for many tasks (including OCT and OCTA image generation) is lower than the processing speed. To reduce data transfer time, B-scans at multiple scan locations (e.g., 12 B-scans at four locations) may be batched for each transfer. With batched transfer of 12 B-scans at four locations, the total processing and transfer time for each batch may be less than 30 milliseconds (ms), lower than the 12 B-scan acquisition time of 42 ms. The OCT and OCTA cross-sectional images are projected using mean projection to generate OCT and OCTA en face images. In embodiments, cross-sectional and/or en face images may be displayed on a custom graphical user interface (GUI) in real-time.

FIG. 1 illustrates a GPU-based OCT/OCTA image processing method 100 (hereinafter “method 100”) in accordance with various embodiments. The method 100 may be performed in real-time to process the associated OCT/OCTA data. The method 100 may be performed by a GPU 102 in conjunction with a CPU 104. Although the method 100 is described herein with reference to a GPU 102 and a CPU 104, other suitable processing circuitry may be used to perform the method 100 in some embodiments.

At 106 of the method 100, the GPU 102 may receive raw data 108 from the CPU 104. The raw data 108 may correspond to an OCT dataset (OCT spectrum) measured on a sample and obtained by an OCT system. At 110, the method 100 may include applying a dispersion compensation algorithm to the raw spectrum. The dispersion compensation algorithm may be applied by the GPU 102. The GPU 102 may generate full OCT spectrum data 112 and split spectrum data 114. The split spectrum data 114 may be generated, for example, using the SSADA method.

At 116 of the method 100, the full OCT spectrum data 112 and split spectrum data 114 may be processed using a Fast Fourier Transform (FFT). The GPU 102 may generate an OCT image 118 from the FFT of the full OCT spectrum data 112. At 120 of the method 100, a decorrelation algorithm may be applied to the FFT of the split spectrum data 114 to obtain decorrelation values. An OCTA image 122 may be generated from the decorrelation values.

At 124 of the method 100, the GPU 102 may provide image data 126 to the CPU 104. The image data 126 may correspond to the OCT image 118 and/or OCTA image 122.

### Self-Tracking OCT/OCTA

Conventional eye motion tracking algorithms are based on the correlation between two sequentially acquired fundus en face images acquired either from infrared camera or SLO. The information used to calculate correlation is mainly from the major retinal vasculatures. This procedure requires additional hardware and software support.

However, OCTA generates vasculature and motion signal itself. Tracking techniques described herein use the OCTA signal for tracking. To better represent the motion strength, an instantaneous MSI may be defined using the normalized standard deviation of the en face OCTA data. For example, the MSI may be determined according to Equation (1):

$$\text{MSI} = \frac{\text{std} \left({D}_{\text{OCTA}}\right)}{\text{mean} \left({D}_{\text{OCTA}}\right)}$$

D_{OCTA} represents the mean projection en face OCTA image values generated from single batched raw data. The MSI may be calculated in a single CPU thread (e.g., four B-frames) after the OCTA en face image is generated in the GPU. The MSI may alternatively or additionally be calculated using the whole batched OCTA volume data (e.g., before the en face image). Additionally, in some embodiments, a normalized variance of the OCTA data may be used instead of or in addition to using standard deviation.

Motion (e.g., eye blink, involuntary motion, and/or microsaccades motion) may be determined when the MSI exceeds a threshold value. The use of normalized values for determination of the MSI may enable a global threshold to be used across patients and/or equipment.

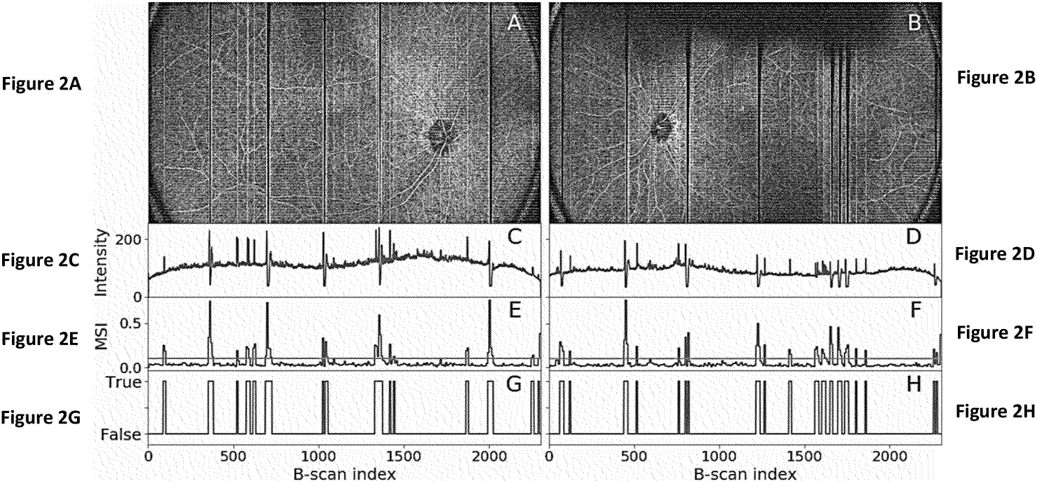

The performance of the MSI was evaluated using wide-field en face OCTA images generated in real-time from a healthy human volunteer (see FIGS. 2A-2H). The threshold is shown by line 202 in FIGS. 2E and 2F.

In various embodiments, blink detection may be performed in addition to and/or instead of the detection of microsaccades, e.g., to achieve artifact-free OCTA imaging. During high-resolution wide-field OCTA image acquisition, the imaging subject can freely blink several times in order to keep the tear film intact. During the blinking time course, the signal strength is significantly reduced. One approach for blink detection is to set a secondary threshold on the signal strength of OCT structural image. When the signal strength is lower than the threshold, a blink may be detected. However, this approach may cause many false detections during wide-field imaging. One of the major artifacts of wide-field OCT image are shadow artifacts caused by vignetting and vitreous opacity (such as floaters), which frequently occur in wide FOV imaging and will significantly lower the signal strength. The signal strength decrease caused by shadow artifacts can be mistakenly detected as a blink, which will fool the tracking system.

However, during blinking, the eye has a relatively large movement in the axial direction. This motion can be accurately detected by MSI. When the eye is closed, because of the low variation in the OCTA signal, the MSI value is low across an entire batch of B-scans. If we combine the batch before the eye closed and the current batch, the MSI value calculated from the combined batch will yield a high value, which indicate blinking artifacts. In the motion and blinking detection mechanism described here, the MSI may be a normalized metric which is independent of the variance of OCTA signal strength and a fixed MSI threshold can be used across different imaging subjects and different systems. After accurately detecting the motion and blink artifacts, the system can then automatically rescan the artifact-affected areas to restore image quality.

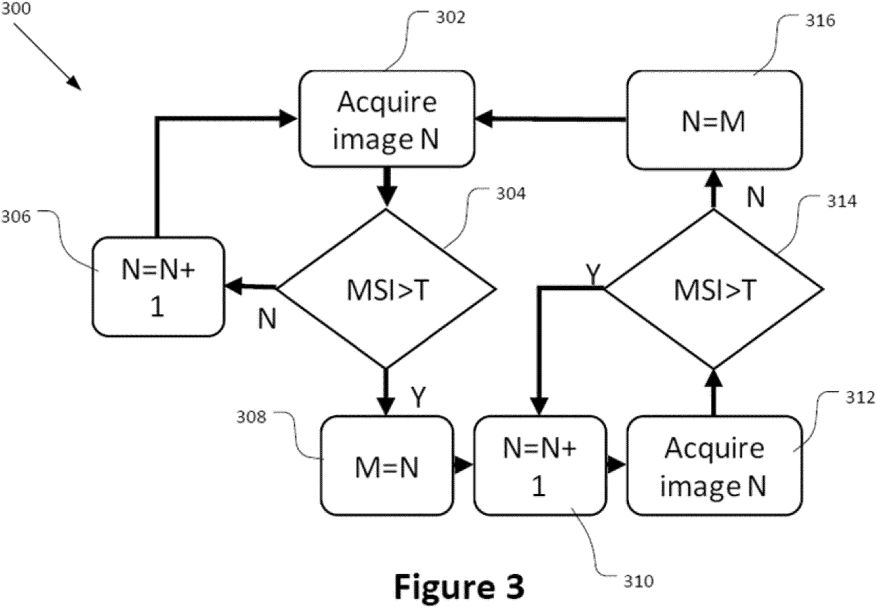

FIG. 3 is a flow chart illustrating a tracking process 300 in accordance with some embodiments. The tracking process 300 may be a real-time self-tracking process. In some embodiments, the tracking process 300 may be performed by a GPU (e.g., GPU 102).

At 302, the method 300 may include acquiring image data for B-scan index N. The image data may be OCTA data, for example, acquired using split spectrum OCT (SS-OCT), such as SSADA. At 304, the method may include calculating the MSI for the image data and comparing it to a threshold T. If the calculated MSI is less than the threshold (e.g., indicating that no excessive motion is detected), the method 300 proceeds to block 306, where the B-scan index N is incremented by one and the method 300 returns to block 302 to acquire image data at the next B-scan index.

However, if the calculated MSI is greater than the threshold T (e.g., indicating that excessive motion is detected), then the method 300 proceeds to a secondary control loop to acquire image data at subsequent B-scan indexes until the excessive motion is no longer detected. For example, the method 300 may record the initial B-scan index N (e.g., as value M) at block 308. At block 310, the B-scan index N may be incremented by 1, and, at block 312, image data at that next B-scan index may be acquired.

At 314, the method 300 may include calculating the MSI for the image data acquired at block 312 and comparing the updated MSI to the threshold T. If the MSI is determined at block 314 to be greater than the threshold (e.g., indicating that excessive motion is still detected), then the method 300 returns to blocks 310 and 312 to acquire image data at the next B-scan index. In this way, the method 300 continues scanning the sample to acquire image data at the following B-scan positions while excessive motion is detected.

When it is determined at block 314 that the MSI is less than the threshold (e..g, indicating that the eye blink and/or micro saccades motion is over), then, at block 316, N is reset to the initial value of the B-scan index that caused the MSI at block 304 to be below the threshold (e.g., N=M). The method 300 then returns to block 302 to re-scan the positions at which excessive motion was detected. Accordingly, the method 300 may perform real-time tracking and correction of artifacts from motion, such as eye blink and/or micro saccades motion.

In some embodiments, after the scanner is reset and the updated data for the region is obtained, the system may calculate the MSI from combined batches of data from before the motion artifact(s) was detected and the updated data acquired after the scanning reset. Accordingly, the quality of the image can be reevaluated.

### X Scan Pattern for Alignment

Wide-field OCT/OCTA image acquisition requires highly accurate alignment. Effectively avoiding vignetting artifacts can increase the image quality. Embodiments herein provide a novel X scanning pattern. The X scanning pattern may show the horizontal and vertical scans at the same time, and may help the OCTA operator align the OCT axis to the eye.

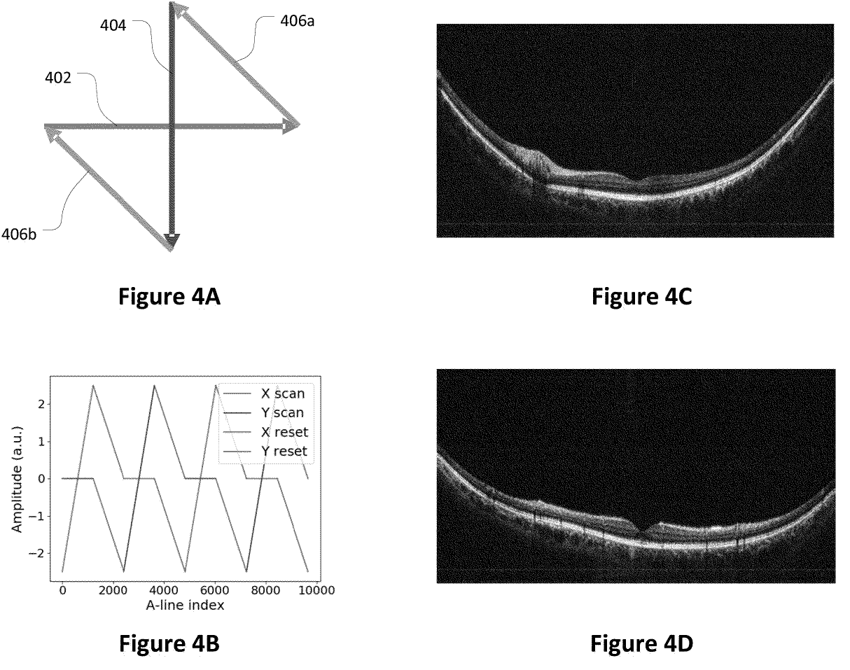

FIG. 4 depicts a scanning pattern 400 in accordance with some embodiments. The scanning pattern 400 includes a horizontal scan 402 and a vertical scan 404. The scanning pattern 400 may further include one or more fly-back scans 406a-b, such as a fly-back scan 406a from the end of the horizontal scan 402 to the beginning of the vertical scan 404 and/or a fly-back scan 406b from the end of the vertical scan to the beginning of the next horizontal scan. Each of these scans 402, 404, 406a, and/or 406b can be used to generate OCT images. In some implementations, only the horizontal scan 402 and the vertical scan 404 are displayed. By using these two orthogonal scans, the OCT operator can effectively adjust the horizontal and vertical position to avoid vignetting artifacts from misalignment.

After performing the cross-scanning pattern for alignment (e.g., using scanning pattern 400), OCT data may be acquired using a different scanning pattern (e.g., a raster scan pattern or a bidirectional scan pattern).

### Example Results

The following examples are illustrative of the disclosed methods. In light of this disclosure, those skilled in the art will recognize that variations of these examples and other examples of the disclosed methods would be possible without undue experimentation.

### Healthy Human

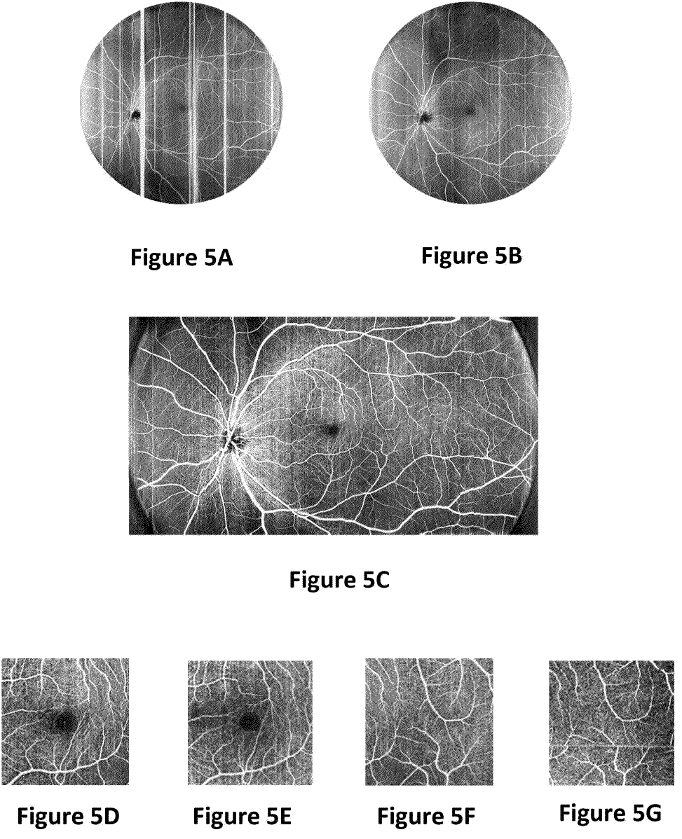

To evaluate performance of the tracking system, the algorithm described herein was applied with different scanning patterns and scanning methods on both a healthy volunteer and a DR patient. First, two wide-field high-resolution OCTA images were acquired from a healthy human volunteer. One image was acquired without the tracking engaged; another image was acquired with the tracking engaged. For each volume, images contain 2560 A-lines per B-scan and 1920 B-scans per volume with two repeats. The sampling step size in the fast axis is 9 µm and in the slow axis is 12 µm. The total data acquisition time is 25 seconds without tracking. In this work, the total scanning time of the image with tracking is less than 1 minute. During the data acquisition with tracking, when the volunteer blinked their eyes, the system successfully detected each blink and rescanned the area right after the eye re-opened. Motion artifacts were also successfully detected each time. Large motion artifacts were successfully removed; however, in order to complete the scan within a reasonable time (e.g., <1 min), some mild motion artifacts that are lower than the MSI threshold are intentionally not detected. The inner retina OCTA image is generated after layer segmentation using maximum projection. A Gabor filter, histogram equalization, and a custom color map were applied for display (see FIGS. 5A and 5B). A higher resolution OCTA image can also be acquired in a smaller field of view.

The same healthy subject’s retinal image was acquired with horizontal 75-degree FOV and vertical 38-degree FOV. The equivalent image size is 23 ×12 mm. The image contains 1208 A-lines per B-scan, 2304 B-scans per volume with 3 repeated B-scans at each cross-sectional location for better signal to noise ratio (SNR). The horizontal and vertical digital resolution is 10 µm. This enabled a higher lateral resolution and SNR. The OCTA image is then generated using the same method mentioned previously (FIG. 5C). In the image, all the blinks and large movements have been avoided. Only a few minor motion artifacts remain. The high scanning density enabled acquisition of high-resolution images with fine vascular details. For image quality comparison, the same healthy human subject was also scanned using a commercial OCT system (Optovue), with 3 × 3 mm retinal images acquired in both the central macular region and the peripheral temporal area. Only the x-fast OCTA scans are used to generate the inner retinal en face images. For comparison, images were cropped at the same position as the commercial scans from the high resolution OCTA image (FIG. 5C). Both the images from the prototype system and the commercial system show similar image quality and capillary visibility.

The performance of the tracking system can also be evaluated quantitatively. Fourteen eyes from seven healthy human subjects were scanned. Both images with tracking and without tracking were acquired. The real-time OCTA en face images were generated along with the OCT spectrum raw data. 104 blinks total were counted across all of these images. After enabling the tracking the total number of blinks visible was reduced to 0. The motion is counted automatically by calculating the MSI, and the threshold is set to be 0.25. MSI larger than 0.25 is considered to be a significant movement. The number of total movements is 1976, after enabling the tracking, the total number of movements was reduced to 168. This means the automated system achieved a 100% and 91.5% reduction rate for blinking and movement, respectively.

### Patients with DR

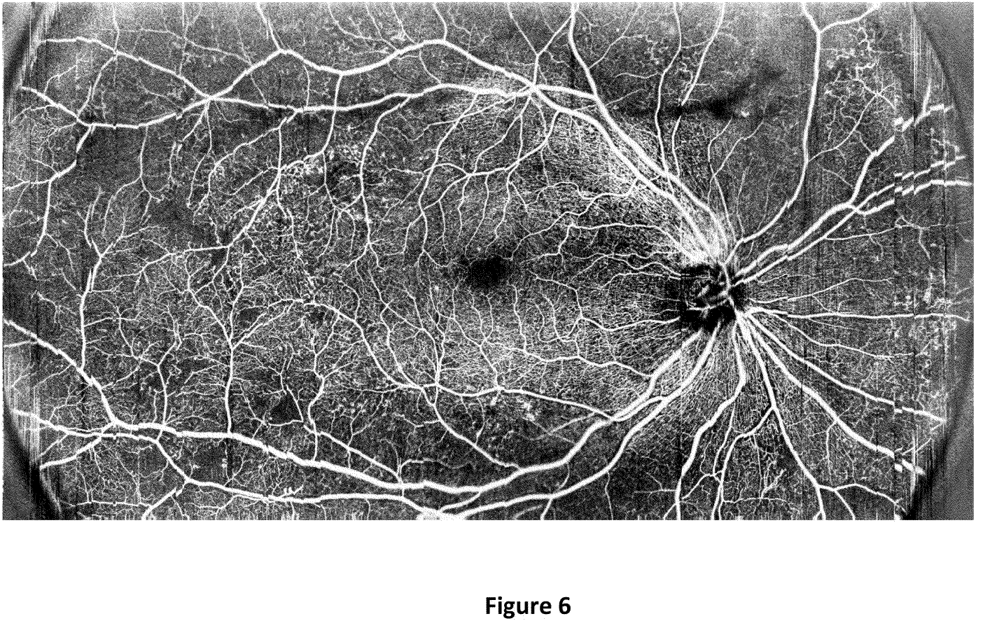

Compared to image acquisition on healthy human subjects, imaging on patients with DR is more challenging. Here, images were acquired on a 57-year-old female diagnosed with proliferative DR and early stage cataract. A 23 × 12 mm retinal image was acquired. Even through the prototype system does not require dilation, to decrease the difficulty of alignment during patient imaging the patient volunteer was pre-dilated before image acquisition. A denser vasculature can be found in the central macular area, however, in the peripheral area, large numbers of non-perfusion areas can be found (FIG. 6). The patient imaging indicates that the tracking system described herein could be useful in clinical applications.

Accordingly, the OCTA-based motion and blink detection and tracking method described herein has been successfully applied to image both healthy and DR-diagnosed human volunteers. This method doesn’t require additional hardware modification to the OCT system. 75-degree wide-field capillary level resolution images have been acquired. It has achieved comparable capillary visibility to commercial OCT system.

The OCTA based real-time self-tracking method described herein provides several advantages over prior techniques. Previously, prototype and commercial OCT systems either used infrared fundus photography or infrared SLO as the reference for eye tracking and blink detection. The additional hardware required by these systems increases the budget and the complexity of the OCT system. There are several advantages of OCTA self-tracking over conventional tracking method. For example, no additional hardware is required; OCTA provides richer vasculature information to evaluate motion amplitude; OCTA is intrinsically sensitive to movements, such as microsaccades, which makes the detection of motion much easier; and/or motion on OCTA can be directly assessed by a self-tracking scheme, which is more reliable than the assessment through third party imaging modalities.

GPU-based data processing technology accelerated the development of real-time OCT/OCTA. The self-tracking method described herein may rely on high-speed real-time OCTA image processing to achieve a fast response to motion and blinking artifacts. The current bottleneck in real-time OCTA image processing is the data transfer speed. For example, the data transfer from the host to GPU may be more time consuming than data processing in the GPU. For real-time applications, the data transfer and processing time should be less than the data acquisition time. For example, in the 400 kHz OCT system with 1204 A-lines per B-scan, a minimum of 12 B-scans may need to be batched and processed together to maintain the real-time processing (e.g., in a Nvidia™ RTX 2080ti GPU). A larger batch will increase the data processing efficiency but also the data transfer time. For a tracking system, a balance between the processing efficiency and the tracking response time may be struck. A large batch will have a longer acquisition and processing time, which will result in a long response time. A slow response tracking system will directly extend the data acquisition time.

As discussed above, the tracking system described herein may use the MSI. As the key motion indicator, the reliability of MSI directly correlated to the reliability of the tracking. Here, MSI may be a normalized value across several different B-scans. The normalization process removes the dependency on signal strength, yielding a pure correlation to motion. Thus, in the tracking algorithm described herein, MSI may be independent to variation between different imaging subjects, different SNR, and/or different types of systems. However, MSI is still affected by the number of B-frames used in each batch. For example, a smaller batch may render an unreliable MSI. In one example system, a high speed swept source laser was used, which requires at least 12 B-scans in each batch.

Compared to imaging of healthy subjects, patients with DR are more challenging, in general requiring both higher speeds and higher sensitivities. Currently, high-speed swept source lasers exist and have been applied to prototype OCT systems. One of the fastest swept wavelength laser sources is the Fourier domain mode lock (FDML) laser. It can achieve megahertz swept source rates. At such a high speed, video-rate OCT imaging can be achieved.

The example system described herein employed a 400 kHz rather than a megahertz swept source laser. There are several considerations when selecting a laser source; one is that increasing sweep rate decreases the SNR of the OCTA system. Another is the scanning speed. High scanning speeds require a resonant scanner. However, resonant scanners cause image distortion problems. Furthermore, high B-scan rates reduce OCTA image quality. Finally, for wide-field imaging, a sufficient imaging range is required. For example, for a three-megahertz OCT system, suppose 1536 pixels per A-line are needed. To acquire those pixels, a 5 GHz balanced detector is needed. However, currently, it is a challenge to design such a balanced detector. A 400 kHz laser source may balance these considerations.

Another challenge for wide-field OCTA imaging is the vignetting. A special optical system was designed to eliminate the shadow caused by the pupil, and further reduced the vignetting problem. Additionally, the system may take advantage of GPU and SSADA processing efficiency. Together, these software and/or hardware improvements may enable the high-quality images presented herein.

The tracking system described herein provides several advantages, but still has some limitations. For example, in some embodiments, the tracking system described herein may only provide an indicator that motion or blinking occurred, and may not provide any quantitative lateral or axial motion information. Without such information, the system is reliant on the fixation target and the cooperation of the patient to realign the eye after the motion. This may introduce artifacts like vessel interruption.

Accordingly, embodiments herein provide a real-time OCTA-based motion and blinking detection system with eye tracking. The self-tracking system may be integrated in a wide-field high-speed swept-source OCT system that acquires a wide-field (e.g., 75-degree field-of-view) high-density OCTA image. Eye blinking and large motion may be successfully detected by calculating the instantaneous motion index and rescanning in real-time. Healthy and DR patient volunteers have been successfully imaged. The field-of-view and the resolution are significantly improved compared to a conventional OCT system.

### Example Optical Coherence Tomography Angiography Image Processing System

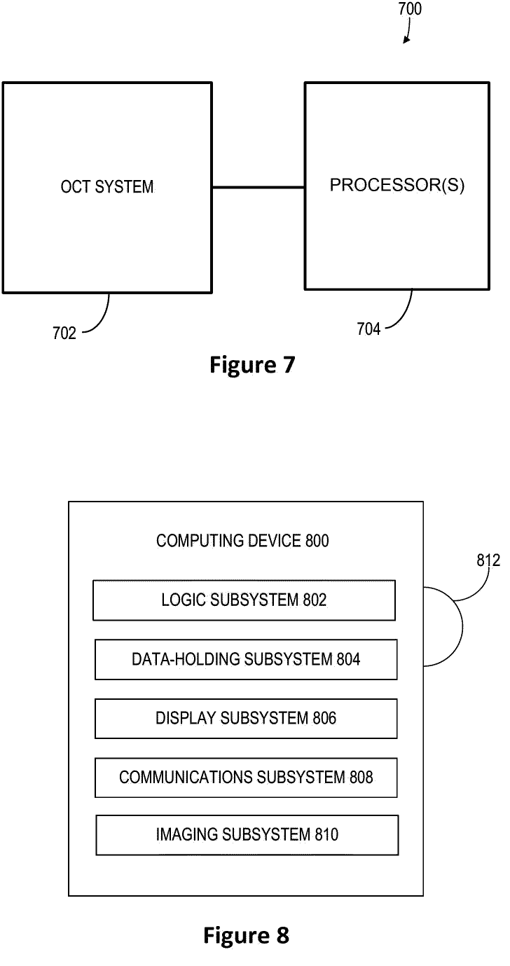

FIG. 7 schematically shows an example system 700 for OCT image processing in accordance with various embodiments. System 700 comprises an OCT system 702 configured to acquire an OCT image comprising OCT interferograms and one or more processors or computing systems 704 that are configured to implement the various processing routines described herein. OCT system 700 can comprise an OCT system suitable for structural OCT and OCT angiography applications, e.g., a swept source OCT system or spectral domain OCT system.

In various embodiments, an OCT system can be adapted to allow an operator to perform various tasks. For example, an OCT system can be adapted to allow an operator to configure and/or launch various ones of the herein described methods. In some embodiments, an OCT system can be adapted to generate, or cause to be generated, reports of various information including, for example, reports of the results of scans run on a sample.

In embodiments of OCT systems comprising a display device, data and/or other information can be displayed for an operator. In embodiments, a display device can be adapted to receive an input (e.g., by a touch screen, actuation of an icon, manipulation of an input device such as a joystick or knob, etc.) and the input can, in some cases, be communicated (actively and/or passively) to one or more processors. In various embodiments, data and/or information can be displayed, and an operator can input information in response thereto.

In some embodiments, the above described methods and processes can be tied to a computing system, including one or more computers. In particular, the methods and processes described herein, e.g., the methods and processes for HDR-OCTA described above, can be implemented as a computer application, computer service, computer API, computer library, and/or other computer program product.

FIG. 8 schematically shows a non-limiting computing device 800 that can perform one or more of the above described methods and processes. For example, computing device 800 can represent a processor included in system 800 described above, and can be operatively coupled to, in communication with, or included in an OCT system or OCT image acquisition apparatus. Computing device 800 is shown in simplified form. It is to be understood that virtually any computer architecture can be used without departing from the scope of this disclosure. In different embodiments, computing device 800 can take the form of a microcomputer, an integrated computer circuit, printed circuit board (PCB), microchip, a mainframe computer, server computer, desktop computer, laptop computer, tablet computer, home entertainment computer, network computing device, mobile computing device, mobile communication device, gaming device, etc.

Computing device 800 includes a logic subsystem 802 and a data-holding subsystem 804. Computing device 800 can optionally include a display subsystem 806, a communication subsystem 808, an imaging subsystem 810, and/or other components not shown in FIG. 8. Computing device 800 can also optionally include user input devices such as manually actuated buttons, switches, keyboards, mice, game controllers, cameras, microphones, and/or touch screens, for example.

Logic subsystem 802 can include one or more physical devices configured to execute one or more machine-readable instructions. For example, the logic subsystem can be configured to execute one or more instructions that are part of one or more applications, services, programs, routines, libraries, objects, components, data structures, or other logical constructs. Such instructions can be implemented to perform a task, implement a data type, transform the state of one or more devices, or otherwise arrive at a desired result.

The logic subsystem can include one or more processors that are configured to execute software instructions. For example, the one or more processors can comprise physical circuitry programmed to perform various acts described herein. Additionally or alternatively, the logic subsystem can include one or more hardware or firmware logic machines configured to execute hardware or firmware instructions. Processors of the logic subsystem can be single core or multicore, and the programs executed thereon can be configured for parallel or distributed processing. The logic subsystem can optionally include individual components that are distributed throughout two or more devices, which can be remotely located and/or configured for coordinated processing. One or more aspects of the logic subsystem can be virtualized and executed by remotely accessible networked computing devices configured in a cloud computing configuration.

Data-holding subsystem 804 can include one or more physical, non-transitory, devices configured to hold data and/or instructions executable by the logic subsystem to implement the herein described methods and processes. When such methods and processes are implemented, the state of data-holding subsystem 804 can be transformed (e.g., to hold different data).

Data-holding subsystem 804 can include removable media and/or built-in devices. Data-holding subsystem 804 can include optical memory devices (e.g., CD, DVD, HD-DVD, Blu-Ray Disc, etc.), semiconductor memory devices (e.g., RAM, EPROM, EEPROM, etc.) and/or magnetic memory devices (e.g., hard disk drive, floppy disk drive, tape drive, MRAM, etc.), among others. Data-holding subsystem 804 can include devices with one or more of the following characteristics: volatile, nonvolatile, dynamic, static, read/write, read-only, random access, sequential access, location addressable, file addressable, and content addressable. In some embodiments, logic subsystem 802 and data-holding subsystem 804 can be integrated into one or more common devices, such as an application specific integrated circuit or a system on a chip.

FIG. 8 also shows an aspect of the data-holding subsystem in the form of removable computer-readable storage media 812, which can be used to store and/or transfer data and/or instructions executable to implement the herein described methods and processes. Removable computer-readable storage media 812 can take the form of CDs, DVDs, HD-DVDs, Blu-Ray Discs, EEPROMs, flash memory cards, USB storage devices, and/or floppy disks, among others.

When included, display subsystem 806 can be used to present a visual representation of data held by data-holding subsystem 804. As the herein described methods and processes change the data held by the data-holding subsystem, and thus transform the state of the data-holding subsystem, the state of display subsystem 806 can likewise be transformed to visually represent changes in the underlying data. Display subsystem 806 can include one or more display devices utilizing virtually any type of technology. Such display devices can be combined with logic subsystem 802 and/or data-holding subsystem 804 in a shared enclosure, or such display devices can be peripheral display devices.

When included, communication subsystem 808 can be configured to communicatively couple computing device 800 with one or more other computing devices. Communication subsystem 808 can include wired and/or wireless communication devices compatible with one or more different communication protocols. As non-limiting examples, the communication subsystem can be configured for communication via a wireless telephone network, a wireless local area network, a wired local area network, a wireless wide area network, a wired wide area network, etc. In some embodiments, the communication subsystem can allow computing device 800 to send and/or receive messages to and/or from other devices via a network such as the Internet.

When included, imaging subsystem 810 can be used acquire and/or process any suitable image data from various sensors or imaging devices in communication with computing device 800. For example, imaging subsystem 810 can be configured to acquire OCT image data, e.g., interferograms, as part of an OCT system, e.g., OCT system 702 described above. Imaging subsystem 810 can be combined with logic subsystem 802 and/or data-holding subsystem 804 in a shared enclosure, or such imaging subsystems can comprise periphery imaging devices. Data received from the imaging subsystem can be held by data-holding subsystem 804 and/or removable computer-readable storage media 812, for example.

It is to be understood that the configurations and/or approaches described herein are exemplary in nature, and that these specific embodiments or examples are not to be considered in a limiting sense, because numerous variations are possible. The specific routines or methods described herein can represent one or more of any number of processing strategies. As such, various acts illustrated can be performed in the sequence illustrated, in other sequences, in parallel, or in some cases omitted. Likewise, the order of the above-described processes can be changed.

The subject matter of the present disclosure includes all novel and nonobvious combinations and subcombinations of the various processes, systems and configurations, and other features, functions, acts, and/or properties disclosed herein, as well as any and all equivalents thereof.

1. A method comprising:
obtaining, from an optical coherence tomography (OCT) system, first OCT data at one or more B-scan locations;
obtaining OCT angiography (OCTA) data for the one or more B-scan locations based on the first OCT data;
calculating a motion strength index (MSI) for the one or more B-scan locations based on the OCTA data;
determining that the calculated MSI is greater than a threshold; and
instructing, based on the determination, the OCT system to acquire second OCT data for the one or more B-scan locations.

2. The method of claim 1, wherein the OCTA data is en face OCTA data.

3. The method of claim 1, wherein the one or more B-scan locations are a batch of multiple B-scan locations.

4. The method of claim 3, wherein the MSI is calculated according to:
$$\text{MSI} = \frac{\text{std} \left({D}_{O C T A}\right)}{\text{mean} \left({D}_{O C T A}\right)}$$
wherein DOCTA corresponds to mean projection en face OCTA values of the OCTA data at the batch of multiple B-scan locations.

5. The method of claim 1, wherein the MSI is normalized in relation to signal strength of the first OCT data.

6. The method of claim 1, wherein the MSI is a first MSI, and wherein the method further comprises:
obtaining additional OCT data at subsequent B-scan locations; and
determining when a second MSI for one of the subsequent B-scan locations is less than the threshold;
wherein the OCT system is instructed to acquire the second OCT data responsive to the determination that the second MSI is less than the threshold.

7. The method of claim 1, further comprising instructing the OCT system to:
perform a horizontal scan of a sample along a horizontal axis with an OCT light source;
perform a fly-back scan with the OCT light source from an end location of the horizontal scan to a beginning location of a vertical scan; and
perform a vertical scan with the OCT light source from the beginning location of the vertical scan along a vertical axis, wherein the vertical axis is perpendicular to the horizontal axis.

8. The method of claim 1, wherein the method is performed by a graphics processing unit (GPU).

9. A scanning method for an optical coherence tomography (OCT) system, the scanning method comprising:
performing a horizontal scan of a sample along a horizontal axis with an OCT light source;
performing a fly-back scan with the OCT light source from an end location of the horizontal scan to a beginning location of a vertical scan; and
performing a vertical scan with the OCT light source from the beginning location of the vertical scan along a vertical axis, wherein the vertical axis is perpendicular to the horizontal axis.

10. The scanning method of claim 9, further comprising simultaneously displaying data from the horizontal scan and the vertical scan to an operator of the OCT system.

11. The scanning method of claim 9, wherein the horizontal scan is a first horizontal scan and the fly-back scan is a first fly-back scan, and wherein the method further comprises performing a second fly-back scan from an end location of the vertical scan to a beginning location of a second horizontal scan.

12. A system for optical coherence tomography (OCT) imaging, the system comprising:
an OCT system to acquire first OCT data at one or more B-scan locations of a sample;
a logic subsystem; and
a data holding subsystem comprising machine-readable instructions stored thereon that are executable by the logic subsystem to:
receive the first OCT data from the OCT system;
obtain OCT angiography (OCTA) data for the one or more B-scan locations based on the first OCT data;
calculate a motion strength index (MSI) for the one or more B-scan locations based on the OCTA data;
determine that the calculated MSI is greater than a threshold; and
instruct, based on the determination, the OCT system to acquire second OCT data for the one or more B-scan locations.

13. The system of claim 12, wherein the OCTA data is en face OCTA data.

14. The system of claim 12, wherein the one or more B-scan locations are a batch of multiple B-scan locations.

15. The system of claim 14, wherein the MSI is calculated according to:
$$\text{MSI} = \frac{\text{std} \left({D}_{O C T A}\right)}{\text{mean} \left({D}_{O C T A}\right)}$$
wherein DOCTA corresponds to mean projection en face OCTA values of the OCTA data at the batch of multiple B-scan locations.

16. The system of claim 12, wherein the MSI is normalized in relation to signal strength of the first OCT data.

17. The system of claim 12, wherein the MSI is a first MSI, and wherein the instructions are further executable by the logic subsystem to:
obtain additional OCT data at subsequent B-scan locations; and
determine when a second MSI for one of the subsequent B-scan locations is less than the threshold;
wherein the OCT system is instructed to acquire the second OCT data responsive to the determination that the second MSI is less than the threshold.

18. The system of claim 12, wherein the instructions are further executable by the logic subsystem to, using the OCT system:
perform a horizontal scan of the sample along a horizontal axis with an OCT light source;
perform a fly-back scan with the OCT light source from an end location of the horizontal scan to a beginning location of a vertical scan; and
perform a vertical scan with the OCT light source from the beginning location of the vertical scan along a vertical axis, wherein the vertical axis is perpendicular to the horizontal axis.

19. The system of claim 12, wherein the logic subsystem is implemented in a graphics processing unit (GPU).
